(19) United States
(12) Patent Application Publication
(10) Pub. No.: US 2013/0079384 A1
(43) Pub. Date: Mar. 28, 2013

(54) Means and Methods for Determining Risk of Cardiovascular Disease

(71) Applicants: Esther Elisa Johanna Maria Creemers, Amsterdam (NL); Sara Johanna Pinto-Sietsma, Amstelveen (NL)

(72) Inventors: Esther Elisa Johanna Maria Creemers, Amsterdam (NL); Sara Johanna Pinto-Sietsma, Amstelveen (NL)

(73) Assignee: Academisch Medisch Centrum bij de Universiteit van Amsterdam, Amsterdam (NL)

(21) Appl. No.: 13/640,714

(22) PCT Filed: Apr. 21, 2011
(86) PCT No.: PCT/NL2011/050275

§ 371 Date: Dec. 12, 2012

(30) Foreign Application Priority Data
Apr. 21, 2010 (EP) 10160635.8

(51) Int. Cl. C12Q 1/68; A61K 31/7105

(52) U.S. Cl. CPC C12Q 1/6837; C12Q 1/686; C12Q 1/6876; A61K 31/7105
USPC 514/44 A; 506/9; 435/6.12; 506/16

(57) ABSTRACT

The invention relates to medicine, in particular to internal medicine and/or cardiology. The present invention provides means and methods for typing a sample and identifying and/or treating a patient suffering from or at risk of suffering from cardiovascular disease by measuring mi RNA present in a sample of said patient. The present invention further provides means and methods for identifying new cardiovascular disease therapies.

### The invention relates to medicine, in particular to internal medicine and/or cardiology.

Cardiovascular disease technically comprises any disease that affects the cardiovascular system. However it is usually used to refer to those diseases related to atherosclerosis.

Cardiovascular disease is the number one cause of death and disability in the United States and most European countries. By the time that heart problems are detected, the underlying cause (atherosclerosis) is usually quite advanced, having progressed for decades. There is, therefore, increased emphasis on preventing atherosclerosis by modifying risk factors, such as healthy eating, exercise and avoidance of smoking.

Coronary disease (or coronary artery disease (CAD)) refers to the failure of coronary circulation to supply adequate circulation to cardiac muscle and surrounding tissue. CAD is a complex disease, which is believed to be caused by many genetic factors, environmental factors, and interactions among these factors. Indeed, many risk factors have been identified for CAD, including smoking, advanced age, male gender, diabetes mellitus, high systolic blood pressure, personal history of angina pectoris, high-fat diet, infectious agents, obesity, increased plasma total and low-density lipoprotein (LDL) cholesterol, increased plasma triglycerides, and decreased plasma high-density lipoprotein (HDL) cholesterol. However, family history is one of the most significant independent risk factor for CAD, and twin studies also suggest that genetic factors contribute to the development of CAD. A high death rate, late-onset characteristics of CAD, and complications by phenocopy in families also present major challenges in genetic dissection of this important disease.

The main cause of CAD is comprised of slowly evolving atherosclerotic plaque formation and acute thrombotic occlusive events, leading to ischemic complications.

Because CAD represents a leading cause of human morbidity and mortality, there is a need for innovative diagnostic and therapeutic strategies. Currently it is difficult to accurately predict whether a subject will develop CAD in the future. Nowadays, this is done by risk calculators, which aren't very precise. Furthermore, the risk calculators known in the art are unable to predict whether a subject has slowly developing plaque formation, or is at risk of acute occlusive events. Several clinical tests can be applied to assess whether a person is suffering from cardiovascular disease, such as narrowing of coronary or peripheral arteries. For instance, electrocardiogram, stress tests (exercise electrocardiogram), echocardiography, blood tests (for abnormal levels of certain fats, cholesterol, sugar, and proteins) are used for diagnostic purposes. However, none of these tests can reliably predict the risk of an individual of suffering from cardiovascular disease, such as coronary or peripheral artery disease. Therefore, there is need for new biomarkers, which can more reliably predict the risk of cardiovascular disease.

It is a goal of the present invention to provide means and methods for typing samples of subjects at risk of suffering from cardiovascular disease and for identifying and/or treating subjects suffering or being at risk of suffering from cardiovascular disease. It is a further goal of the invention to provide a method for identifying medicaments useful in cardiovascular disease therapy and for monitoring such therapy.

The present invention provides means and methods for typing a sample and identifying and/or treating a patient suffering from or at risk of suffering from cardiovascular disease by measuring miRNA present in a sample of said patient. The present invention further provides means and methods for identifying new cardiovascular disease therapies.

The invention for the first time shows that altered expression levels of miRNAs in platelets is correlated with cardiovascular disease. Many have searched for genetic biomarkers of cardiovascular disease, but have never done so using platelets. It has for instance been shown that circulating miRNAs are useful biomarkers for the diagnosis of AMI²⁻⁴. Both miR-1 and miR-208 were reported to be elevated in plasma following myocardial injury. However, it is likely that these miRNAs are released from damaged cardiac cells into the blood stream, because for instance the level of miR-1 returned to normal in discharged AMI patients. In contrast to these studies, which investigated circulating miRNAs in the acute phase of cardiovascular disease, the present inventors investigated miRNAs present in platelets in the stable phase of cardiovascular disease, wherein miRNAs released from for instance damaged cardiac cells do not play a role anymore. Further, the miRNAs found by the present inventors have not been found in earlier studies because prior to the present invention, others searching for miRNAs related to cardiovascular disease did not look for miRNAs levels in platelet-purified samples (e.g. platelet rich plasma). Instead miRNA levels were determined in for instance plasma samples during acute cardiovascular disease. During acute cardiovascular disease, however, several miRNAs are up- and/or down-regulated (i.e. dysregulated) in a whole blood or plasma sample. These dysregulated miRNAs include miRNAs released from damaged heart cells, but also for instance from activated white blood cells that act on the heart damage. Also ischemic insults that normally accompany an acute cardiovascular disease have pronounced effects on the expression levels of several miRNAs in blood or plasma. The miRNAs identified as predictive risk factors for cardiovascular disease in the present invention were not identified in any prior study because these miRNAs are not present in whole blood in the acute phase of cardiovascular disease at levels high enough to allow detection of the 1.5-2 fold differences between CAD patients and healthy controls by the screening method used by the inventors (microarrays), which was required to test the large number of total miRNAs.

By using a sample relatively pure in platelets, i.e. a platelet rich plasma sample, the inventors were able to identify miRNAs that are present in platelets and that are dysregulated in subjects at risk but not yet suffering from an acute cardiovascular disease. These miRNA serve as biomarkers for subjects at risk of suffering from cardiovascular disease and can thus be used as predictive biomarkers.

miRNAs are small nucleic acid molecules that suppress protein synthesis by inhibiting mRNA translation or promoting mRNA degradation. Next to being important regulators of normal development and physiology, dysregulated expression of miRNAs has been correlated with a number of human diseases. As a consequence, human miRNAs are considered to be highly useful as biomarkers. Recently, the existence of a microRNA pathway in anucleate platelets has been discovered¹. The present invention now provides the insight that the expression profile of platelet miRNA is useful in determining whether a subject is suffering from, or at risk of suffering from cardiovascular disease. The present invention shows for instance that specific miRNAs are differentially expressed in platelets of patients with CAD when compared to platelets of healthy controls.

In a first embodiment, the invention provides a method for determining whether a subject is at risk of suffering from a cardiovascular disease, the method comprising determining a (relative) level of expression of at least one miRNA using a sample of said subject, said miRNA being selected from the group consisting of miR-545:9.1, miR-624*, and miR-454*, hsa-miR-340*, hsa-miR-615-5p, hsa-miR-451, hsa-miR-1280, and homologues and orthologues of any of these miRNAs, and comparing the level of expression of said at least one miRNA with a reference value, wherein the result of said comparison is indicative of whether said individual is suffering from, or is at risk of suffering from, a cardiovascular disease. Preferably, said method is an in vitro method. Said reference value is preferably obtained by determining the expression level of said at least one miRNA using a reference sample, preferably from a subject not suffering from, or at risk of suffering from, a cardiovascular disease. In a preferred embodiment, said miRNA is selected from the group consisting of hsa-miR-340*, miR-624*, miR-454, and homologues and orthologues of any of these miRNAs. In a more preferred embodiment, said miRNA is hsa-miR-340* or a homologue or orthologue of hsa-miR-340*. In another more preferred embodiment, said miRNA is miR-624* or a homologue or orthologue of miR-624*.

It is not necessary to determine both the reference value and the test value at the same time. It is for instance possible to determine a reference value, preferably using a sample from a subject not suffering from, or at risk of suffering from, a cardiovascular disease, and use said reference value over and over again to determine whether or not the relative expression of said at least one miRNA in a test sample is indicative for an individuals risk of suffering from cardiovascular disease. It is possible to use as a reference value a level of expression that has been shown to be indicative for a low risk of suffering from a cardiovascular disease, for instance the mean level of miRNA in a number of samples from healthy individuals. On the other hand it is also possible to use as a reference value a level of expression that has been shown to be indicative for cardiovascular disease or for a high risk of suffering from a cardiovascular disease. Such reference value is for instance obtained using a sample of an individual suffering from a cardiovascular disease. It is thus possible to determine whether a subject is at risk of suffering from a cardiovascular disease, by comparing the level of expression of any of the above mentioned miRNAs in a sample of said subject with the level of expression in a sample of a healthy individual or with the level of expression in a sample of an individual suffering from a cardiovascular disease. A level of expression which is comparable with that of a healthy individual is indicative for said subject being not at risk of cardiovascular disease. A level of expression which is comparable with that of an individual suffering from cardiovascular disease is indicative for said subject being at risk of suffering from cardiovascular disease. With comparable is meant that the levels of expression are preferably less than 1.8 fold different, more preferably less than 1.5 fold different, more preferably less than 1.3 fold different, most preferably less than 1.2 fold different.

It is preferred that the expression levels of at least two miRNAs selected from the group consisting of miR-545:9.1, miR-624*, and miR-454*, hsa-miR-340*, hsa-miR-615-5p, hsa-miR-451, hsa-miR-1280, and homologues and orthologues of any of these miRNAs are determined in a method according to the invention. It is preferred that at least one of said at least two miRNAs is hsa-miR-340* or miR-624*, or a homologue or orthologue of hsa-miR-340* or miR-624*. More preferably said at least one of said at least two miRNAs is hsa-miR-340* or a homologue or orthologue of hsa-miR-340*. In a preferred embodiment, a first of said at least two miRNAs is hsa-miR-340 or a homologue or orthologue of hsa-miR-340 and a second of said at least two miRNAs is miR-454 or a homologue or orthologue of miR-454. More preferably the levels of expression of at least three, more preferably at least four, more preferably at least five, more preferably at least six, more preferably at least seven, most preferably at least eight miRNAs selected from the group consisting of miR-545:9.1, miR-624*, and miR-454*, hsa-miR-340*, hsa-miR-615-5p, hsa-miR-451 , hsa-miR-1280, and homologues and orthologues of any of these miRNAs are determined in a method according to the invention. Determining several of the here above mentioned miRNAs in a sample of a subject preferably leads to a more accurate assessment of the subject's risk on suffering from cardiovascular disease.

The invention for instance shows that if the expression level of hsa-miR-340* or a homologue or orthologue of hsa-miR-340* in combination with the expression level of at least one other miRNA of the invention is determined, preferably the expression level of miR-624* or a homologue or orthologue of miR-624* and/or that of miR-454* or a homologue or orthologue of miR-454*, a subject's risk on suffering from cardiovascular disease can be measured more accurately than if the expression level of only one of these miRNAs is measured. The same holds true for determining the expression level of miR-624*, or a homologue or orthologue of miR-624* in combination with the expression level of at least one other miRNA of the invention, preferably the expression level of hsa-miR-340* or a homologue or orthologue of hsa-miR-340* and/or that of miR-454* or a homologue or orthologue of miR-454*.

In a preferred embodiment, therefore, a method according to the invention is provided, wherein a (relative) level of expression of at least two miRNAs is determined, wherein a first of said at least two miRNAs is hsa-miR-340* or a homologue or orthologue of hsa-miR-340*, and wherein a second of said at least two miRNAs is selected from the group consisting of miR-624*, miR-454*, miR-545:9.1, hsa-miR-615-5p, hsa-miR-451, hsa-miR-1280, and homologues and orthologues of any of these miRNAs, the method further comprising comparing the level of expression of said at least two miRNA with reference values, wherein the result of said comparison is indicative of whether said individual is at risk of suffering from a cardiovascular disease.

In a more preferred embodiment, a method according to the invention is provided, wherein said second miRNA is selected from the group consisting of miR-624*, miR-454*, hsa-miR-451, and homologues and orthologues of any of these miRNAs. In an even more preferred embodiment, said second miRNA is miR-454* or a homologue or orthologue of miR-454*.

In a most preferred embodiment, a method according to the invention is provided, wherein said second miRNA is miR-624* or a homologue or orthologue of miR-624*, said method further comprising determining a (relative) level of expression of miR-454 or a homologue or orthologue of miR-454, and comparing the level of expression of said first, second and further miRNA with reference values, wherein the result of said comparison is indicative of whether said individual is at risk of suffering from a cardiovascular disease.

In another preferred embodiment, a method according to the invention is provided, wherein a (relative) level of expression of at least two miRNAs is determined, wherein a first of said at least two miRNAs is miR-624* or a homologue or orthologue of miR-624*, and wherein a second of said at least two miRNAs is selected from the group consisting of hsa-miR-340*, miR-454*, miR-545:9.1, hsa-miR-615-5p, hsa-miR-451, hsa-miR-1280, and homologues and orthologues of any of these miRNAs, the method further comprising comparing the level of expression of said at least two miRNA with reference values, wherein the result of said comparison is indicative of whether said individual is at risk of suffering from a cardiovascular disease.

In a more preferred embodiment, a method according to the invention is provided, wherein said second miRNA is selected from the group consisting of hsa-miR-340*, miR-454*, hsa-miR-451, and homologues and orthologues of any of these miRNAs.

With a “healthy subject” or “a subject not suffering from, or at risk of suffering from, a cardiovascular disease” is meant a subject which does not have an increased risk relative to the normal population. Preferably such subjects do not have overweight or high cholesterol, are non-smokers, non-diabetic and do not have a history or family history of cardiovascular disease.

With a “subject suffering from, or at risk of suffering from, a cardiovascular disease” is meant a subject which is already diagnosed with cardiovascular disease and/or has an increased risk relative to the normal population of suffering from cardiovascular disease.

The level of expression of at least one miRNA present in platelets is preferably determined using a sample comprising said platelets. It is, however, not necessary that the platelets in the sample are still intact and/or the miRNA is still present within the platelet in order to determine the level of expression thereof. For instance, the platelets may be, and preferably are, lysed before determining the level of miRNA of the invention. The amount of miRNA measured in said sample is then used to calculate a (relative) level of said miRNA present in said platelet. The word “relative” is used because it is not necessary to determine an absolute value (e.g. in pg/1×10⁶ platelets), but the level is preferably determined relative to a housekeeping gene, such as RNU6B, a widely used endogenous reference RNA in many miRNA quantification studies.

It is noted that the invention has identified several miRNAs that are differentially expressed in platelets of individuals at risk of a cardiovascular disease as compared to the expression in platelets in healthy individuals, using a relative platelet pure sample, i.e. a platelet rich plasma sample. Now that the invention has identified several miRNAs differentially expressed in platelets, it is not necessary to use platelet enriched samples in a method according to the invention. For instance, a whole blood sample can be used in a method of the invention for determining whether an individual is at risk of suffering from a cardiovascular disease.

It is preferably avoided, however, that preparation of a sample for use in a method according to the invention induces changes in miRNA levels in said platelet and/or in said sample. This is for instance the case if a blood sample is drawn without an anti-coagulant. The handling of such a blood sample induces activation of platelets and subsequent coagulation. Coagulation will lead to alteration of miRNA levels in the sample.

A homologue of an miRNA is herein defined as an miRNA which is similar due to shared ancestry. The DNA sequences are usually similar, not identical.

An orthologue of an miRNA is herein defined as a homologous nucleic acid molecule, separated by a speciation event. Orthologous genes, for instance, are genes in different species that are similar to each other because the species originated from a common ancestor. It is of course preferred to use an miRNA orthologue from a specific species for diagnosis and/or treatment of a cardiovascular disease in said specific species. Thus in humans, when determining miRNA in a human sample, human miRNA is preferably determined.

In a preferred embodiment, the homologue and/or orthologue has at least 50% sequence identity with an miRNA of the invention. In a more preferred embodiment, the sequence identity is at least 60%, more preferably at least 70%, more preferably at least 80%, more preferably at least 90%, most preferably at least 95%.

The term “% sequence identity” is defined herein as the percentage of nucleotides in a nucleic acid sequence that is identical with the nucleotides in a nucleic acid sequence of interest, after aligning the sequences and optionally introducing gaps, if necessary, to achieve the maximum percent sequence identity. Methods and computer programs for alignments are well known in the art.

Because the level of expression of miRNA in platelets is determined, it is preferred that a sample is used that comprises platelets, for instance a blood sample or a platelet rich plasma sample. In a preferred embodiment, therefore, a method according to the invention is provided, wherein a whole blood sample is used, preferably a blood sample enriched for platelets. With whole blood sample is meant a venous or arterial blood sample, preferably drawn in an open system (i.e. without vacuum) and preferably with an anti-coagulant. Vacuum is known to stress the blood cells and may lead to activation of platelets and subsequent alteration of miRNA levels in the sample. The use of an anti-coagulant prevents and/or reduces coagulation of blood, and thus prevents and/or reduces coagulation induced changes in miRNA levels in the sample. In an even more preferred embodiment, a method according to the invention is provided, wherein said sample is a platelet rich plasma sample.

Platelet rich plasma is herewith defined as plasma with a platelet concentration above baseline (i.e. platelet concentration in normal plasma). Methods for preparing platelet rich plasma by centrifugation are known by the skilled person. Platelet rich plasma may or may not also contain increased concentrations of white blood cells. In a method according to the invention, however, it is preferred to use a platelet rich plasma sample with a low number of white blood cells. Preferably said sample comprises below 5%, more preferably below 2.5%, more preferably below 1%, even more preferably below 0.5%, most preferably below 0.4% white blood cells, relative to the amount of platelets in said sample.

According to the present invention, from the miRNAs that are differentially expressed in CAD patients versus controls, hsa-miR-1280 is down-regulated and hsa-miR-340*, hsa-miR-615-5p, hsa-miR-545:9.1, hsa-miR-451, hsa-miR-454*, and hsa-miR-624* are up-regulated in CAD patients. Because of variability of test results and inter-individual differences, it is defined herein that a 1.5 fold down- or up-regulation is considered significant and predictive for whether a person is at risk of suffering from cardiovascular disease.

In a preferred embodiment, therefore, a method according to the invention is provided, comprising determining whether the level of expression of said at least one miRNA in said sample is at least 1.50 fold increased, relative to the level of expression of said at least one miRNA in a sample of a healthy individual not at risk of cardiovascular disease, wherein said at least one miRNA comprises an miRNA selected from the group consisting of miR-545:9.1, miR-624*, and miR-454*, hsa-miR-340*, hsa-miR-615-5p, hsa-miR-451, and homologues and orthologues of any of these miRNAs. In a more preferred embodiment, it is determined whether said level of expression is at least 1.55, preferably at least 1.60, more preferably at least 1.65, more preferably at least 1.75, most preferably at least 1.85 fold increased.

In another preferred embodiment, a method according to the invention is provided, comprising determining whether the level of expression of said at least one miRNA in said sample is at least 1.50 fold decreased relative to the level of expression of said at least one miRNA in a sample of a healthy individual not at risk of cardiovascular disease, wherein said miRNA is hsa-miR-1280 or a homologue or orthologue thereof. In a more preferred embodiment, it is determined whether said level of expression is at least 1.55, more preferably at least 1.60, more preferably at least 1.65, more preferably at least 1.75, most preferably at least 1.85 fold decreased.

In a preferred embodiment, a method according to the invention is provided, wherein said at least one miRNA is selected from the group consisting of miR-545:9.1, miR-624*, and miR-454*, hsa-miR-340*, hsa-miR-615-5p, and homologues and orthologues of any of these miRNAs. In a more preferred embodiment, a method according to the invention is provided, wherein said at least one miRNA is selected from the group consisting of miR-545:9.1, miR-624*, miR-454*, and homologues and orthologues of any of these miRNAs.

In a preferred embodiment, a method according to the invention is provided, comprising determining whether the level of expression of hsa-miR-340* and/or a homologue and/or orthologue of hsa-miR-340* in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of hsa-miR-340* and/or a homologue and/or orthologue of hsa-miR-340* in a sample of a healthy individual not at risk of cardiovascular disease, and determining whether the level of expression of a second miRNA in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of said second miRNA in a sample of a healthy individual not at risk of cardiovascular disease, wherein said second miRNA is selected from the group consisting of miR-545:9.1, miR-624*, and miR-454*, hsa-miR-615-5p, and homologues and orthologues of any of these miRNAs. In a more preferred embodiment, said second miRNA is selected from the group consisting of miR-624*, and miR-454*, and homologues and orthologues of any of these miRNAs. In a more preferred embodiment, said second miRNA is miR-454* or a homologue or orthologue of miR-454*.

In a more preferred embodiment, a method according to the invention is provided, comprising determining whether the level of expression of hsa-miR-340* and/or a homologue and/or orthologue of hsa-miR-340* in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of hsa-miR-340* and/or a homologue and/or orthologue of hsa-miR-340* in a sample of a healthy individual not at risk of cardiovascular disease, determining whether the level of expression of miR-624* and/or a homologue and/or orthologue of miR-624* in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of miR-624* and/or a homologue and/or orthologue of miR-624* in a sample of a healthy individual not at risk of cardiovascular disease, and determining whether the level of expression of miR-454 and/or a homologue and/or orthologue of miR-454 in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of miR-454 and/or a homologue and/or orthologue of miR-454 in a sample of a healthy individual not at risk of cardiovascular disease.

In another preferred embodiment, a method according to the invention is provided, comprising determining whether the level of expression of miR-624* and/or a homologue and/or orthologue of miR-624* in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of miR-624* and/or a homologue and/or orthologue of miR-624* in a sample of a healthy individual not at risk of cardiovascular disease, and determining whether the level of expression of a second miRNA in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold increased relative to the level of expression of said second miRNA in a sample of a healthy individual not at risk of cardiovascular disease, wherein said second miRNA is selected from the group consisting of miR-545:9.1, hsa-miR-340*, and miR-454*, hsa-miR-615-5p, and homologues and orthologues of any of these miRNAs. In a more preferred embodiment, said second miRNA is selected from the group consisting of hsa-miR-340*, and miR-454*, and homologues and orthologues of any of these miRNAs.

In a preferred embodiment, the invention also provides a method according to the invention, further comprising determining whether the level of expression of hsa-miR-1280 or a homologue or orthologue thereof in said sample is at least 1.15 fold, preferably at least 1.2, more preferably at least 1.25, more preferably at least 1.3, more preferably at least 1.4, more preferably at least 1.5, more preferably at least 1.60, more preferably at least 1.65, more preferably at least 1.75, most preferably at least 1.85 fold decreased relative to the level of expression of hsa-miR-1280 or a homologue or orthologue thereof in a sample of a healthy individual not at risk of cardiovascular disease.

miRNAs are not mere indicators for whether a person is at risk of suffering from cardiovascular disease. As said before, miRNA are able to suppress protein synthesis by inhibiting mRNA translation or by promoting mRNA degradation. miRNAs are thus potent regulators of normal development and physiology. Increased expression of miRNAs will lead to suppressed protein syntheses of their target. Increased expression of hsa-miR-340*, hsa-miR-615-5p, hsa-miR-545:9.1, hsa-miR-451, hsa-miR-454*, and/or hsa-miR-624* was observed in subjects at risk of suffering from cardiovascular disease. For treating, diminishing and or preventing cardiovascular disease, it is thus useful to counteract the action of such increased miRNA for instance by increasing the amount, expression and/or activity of the target of any of these miRNAs. Increasing the amount, expression and/or activity of an miRNA target can be done for instance by inhibiting the amount, activity and/or expression of said miRNA or by providing the target itself.

In one embodiment therefore, the invention provides a method for treating, diminishing, delaying and/or preventing cardiovascular disease, comprising increasing the amount, expression and/or activity of a target of at least one miRNA selected from the group consisting of miR-545:9.1, miR-624*, and miR-454*, hsa-miR-340*, hsa-miR-615-5p, hsa-miR-451, and homologues and orthologues of any of these miRNAs in a platelet and/or in a megakaryocyte of a subject suffering from or at risk of suffering from said disease. Preferably, said at least one miRNA is selected from the group consisting of miR-545:9.1, miR-624*, and miR-454*, hsa-miR-340*, hsa-miR-615-5p, and homologues and orthologues of any of these miRNAs. More preferably, said at least one miRNA is selected from the group consisting of miR-545:9.1, miR-624*, miR-454*, and homologues and orthologues of any of these miRNAs. In a more preferred embodiment, said at least one miRNA is hsa-miR-340* or miR-624* or a homologue or orthologue of hsa-miR-340* or miR-624*. In an even more preferred embodiment, said at least one miRNA is hsa-miR-340* or a homologue or orthologue of hsa-miR-340*.

The invention provides the insight that increasing the amount, expression and/or activity of one or more targets of at least two, more preferably at least three, more preferably at least four, or more miRNAs selected from the group consisting of miR-545:9.1, miR-624*, and miR-454*, hsa-miR-340*, hsa-miR-615-5p, hsa-miR-451, and homologues and orthologues of any of these miRNAs in a platelet and/or in a megakaryocyte of a subject suffering from or at risk of suffering from said disease is useful in treating diminishing, delaying and/or preventing cardiovascular disease. Especially a combination of at least two miRNAs selected from the group of hsa-miR-340*, miR-624*, miR-454*, and homologues and orthologues of any of these miRNAs is especially useful for treating diminishing, delaying and/or preventing cardiovascular disease. According to the invention increasing the amount, expression and/or activity of one or more targets of hsa-miR-340*, miR-624*, and miR-454*, or homologues and orthologues of any of these miRNAs is most effective in treating diminishing, delaying and/or preventing cardiovascular disease.

In a preferred embodiment, therefore, a method for treating, diminishing, delaying and/or preventing cardiovascular disease according to the invention is provided, wherein said at least one miRNAs is hsa-miR-340* or a homologue or orthologue of hsa-miR-340*, the method further comprising increasing the amount, expression and/or activity of a target of a further miRNA selected from the group consisting of miR-624*, miR-454*, miR-545:9.1, hsa-miR-615-5p, hsa-miR-451, and homologues and orthologues of any of these miRNAs in a platelet and/or in a megakaryocyte of a subject suffering from or at risk of suffering from said disease.

In a more preferred embodiment, a method for treating, diminishing, delaying and/or preventing cardiovascular disease according to the invention is provided, wherein said further miRNA is selected from the group consisting of miR-624*, miR-454*, hsa-miR-451, and homologues and orthologues of any of these miRNAs. In an even more preferred embodiment, said further miRNA is miR-454* or a homologue or orthologue of miR-454.

In a most preferred embodiment, a method for treating, diminishing, delaying and/or preventing cardiovascular disease according to the invention is provided, wherein said further miRNA is miR-624* or a homologue or orthologue of miR-624*, said method further comprising increasing the amount, expression and/or activity of a target of miR-454 or of a target of a homologue or orthologue of miR-454 in a platelet and/or in a megakaryocyte of a subject suffering from or at risk of suffering from said disease.

In another preferred embodiment, a method for treating, diminishing, delaying and/or preventing cardiovascular disease according to the invention is provided, wherein said at least one miRNAs is miR-624* or a homologue or orthologue of miR-624*, the method further comprising increasing the amount, expression and/or activity of a target of a further miRNA selected from the group consisting of hsa-miR-340*, miR-454*, miR-545:9.1, hsa-miR-615-5p, hsa-miR-451, and homologues and orthologues of any of these miRNAs in a platelet and/or in a megakaryocyte of a subject suffering from or at risk of suffering from said disease.

In a more preferred embodiment, a method for treating, diminishing, delaying and/or preventing cardiovascular disease according to the invention is provided, wherein said further miRNA is selected from the group consisting of hsa-miR-340*, miR-454*, hsa-miR-451, and homologues and orthologues of any of these miRNAs.

In a preferred embodiment, a method a for treating, diminishing, delaying and/or preventing cardiovascular disease according to the invention is provided, wherein said increasing the amount, expression and/or activity of said target of miRNA is mediated through inhibition of the expression or activity of said miRNA in said platelet and/or in said megakaryocyte.

Also provided is a method for treating, diminishing, delaying and/or preventing cardiovascular disease, comprising increasing the amount, expression and/or activity of one target or more targets of at least three miRNAs selected from the group consisting of miR-545:9.1, miR-624*, and miR-454*, hsa-miR-340*, hsa-miR-615-5p, hsa-miR-451, and homologues and orthologues of any of these miRNAs in a platelet and/or in a megakaryocyte of a subject suffering from or at risk of suffering from said disease. Preferably, said at least three miRNAs are selected from the group consisting of miR-624*, and miR-454*, hsa-miR-340*, and homologues and orthologues of any of these miRNAs. Most preferably a first of said at least three miRNAs is miR-624* or a homologue or orthologue of miR-624*, a second of said at least three miRNAs is miR-454* or a homologue or orthologue of miR-454*, and a third of said at least three miRNAs is hsa-miR-340* or a homologue or orthologue of miR-340*.

In a preferred embodiment said increasing the amount, expression and/or activity of said target of miRNA is mediated through inhibition of the expression or activity of at least one miRNA selected from the group consisting of miR- 545:9.1, miR-624*, and miR-454*, hsa-miR-340*, hsa-miR-615-5p, hsa-miR-451, and homologues and orthologues of any of these miRNAs in said platelet and/or in said megakaryocyte. Preferably, said at least one miRNA is selected from the group consisting of miR-545:9.1, miR-624*, and miR-454*, hsa-miR-340*, hsa-miR-615-5p, and homologues and orthologues of any of these miRNAs. More preferably, said at least one miRNA is selected from the group consisting of miR-545:9.1, miR-624*, miR-454*, and homologues and orthologues of any of these miRNAs.

Preferably, said inhibition of expression and/or of activity of said miRNA is through miRNA antisense molecules. miRNA antisense molecules are nucleic acid molecules that have a high percentage of complementary sequence identity to an miRNA. The high complementary sequence identity allows for specific binding to said miRNA. With complementary is meant the specific pairing of the purines and pyrimidines between the miRNA and the miRNA antisense molecule. Preferably, said complementary sequence identity of said miRNA antisense molecule with an miRNA of the invention is at least 50%. In a more preferred embodiment, the sequence identity is at least 60%, more preferably at least 70%, more preferably at least 80%, more preferably at least 90%, most preferably at least 95%.

Decreased expression of an miRNA, such as for instance observed for hsa-miR-1280 in subjects suffering from CAD, will lead to increased expression of the miRNA target. The effect of decreased expression of an miRNA can thus be counteracted by decreasing the amount, expression and/or activity of the miRNA target. This can be done for instance by increasing the amount, expression and/or activity of said miRNA.

In a preferred embodiment, therefore, a method for treating, diminishing, delaying and/or preventing cardiovascular disease according to the invention is provided, further comprising decreasing the amount, expression and/or activity of a target of hsa-miR-1280 and/or a homologue and/or orthologue thereof in a platelet and/or megakaryocyte of a subject suffering from or at risk of suffering from said disease. Preferably, said decreasing the amount, expression and/or activity of said target of hsa-miR-1280, and/or homologue and/or orthologue thereof is mediated through increasing the amount, expression and/or activity of hsa-miR-1280, and/or a homologue and/or orthologue and/or analogue thereof in said platelet and/or in said megakaryocyte. In a more preferred embodiment, said decreasing the amount, expression and/or activity of said target of hsa-miR-1280, and/or homologue and/or orthologue thereof is mediated through providing hsa-miR-1280, and/or a homologue and/or orthologue and/or analogue to said subject.

In another embodiment, therefore, a method for treating, diminishing, delaying and/or preventing cardiovascular disease is provided, comprising decreasing the amount, expression and/or activity of a target of hsa-miR-1280 and/or a homologue and/or orthologue thereof in a platelet and/or megakaryocyte of a subject suffering from or at risk of suffering from said disease. Preferably, said decreasing the amount, expression and/or activity of said target of hsa-miR-1280, and/or homologue and/or orthologue thereof is mediated through increasing the amount, expression and/or activity of hsa-miR-1280, and/or a homologue and/or orthologue and/or analogue thereof in said platelet and/or in said megakaryocyte. In a more preferred embodiment, said decreasing the amount, expression and/or activity of said target of hsa-miR-1280, and/or homologue and/or orthologue thereof is mediated through providing hsa-miR-1280, and/or a homologue and/or orthologue and/or analogue to said subject.

In a preferred embodiment, a method according to the invention for treating, diminishing, delaying and/or preventing cardiovascular disease is provided, wherein said homologue, orthologue, and/or analogue of an miRNA has at least 50% sequence identity with said miRNA. In a more preferred embodiment, the sequence identity is at least 60%, at least 70%, at least 80%, at least 90% or at least 95%.

An analogue of an miRNAs is herewith defined as an artificially modified miRNA, comprising one or more residues that are modified, for instance to increase nuclease resistance, and/or to increase the affinity of the antisense nucleotide for the target sequence.

In a preferred embodiment, the analogue comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones.

It is further preferred that the linkage between the residues in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred analogue comprises a Peptide Nucleic Acid (PNA), having a modified polyamide backbone. PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively.

A further preferred backbone comprises a morpholino analogue, in which the ribose or deoxyribose sugar is replaced by a 6-membered morpholino ring. A most preferred miRNA analogue or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, an miRNA analogue of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred miRNA analogue of the invention comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, or aralkyl, that may be interrupted by one or more heteroatoms; O-, S-, or N-alkyl; O-, S-, or N-alkenyl; O-, S-or N-alkynyl; O-, S-, or N-allyl; O-alkyl-O-alkyl, -methoxy, -aminopropoxy; methoxyethoxy; -dimethylaminooxyethoxy; and -dimethylaminoethoxyethoxy. The sugar moiety can be a pyranose or derivative thereof, or a deoxypyranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid. These substitutions render the nucleotide analogue or equivalent RNase H and nuclease resistant and increase the affinity for the target RNA.

In another embodiment, an miRNA analogue of the invention comprises one or more base modifications or substitutions. Modified bases comprise synthetic and natural bases such as inosine, xanthine, hypoxanthine and other aza, deaza, hydroxy, halo, thio, thiol, alkyl, alkenyl, alkynyl, thioalkyl derivatives of pyrimidine and purine bases that are or will be known in the art.

It is understood by a skilled person that it is not necessary for all positions in an antisense oligonucleotide to be modified uniformly. In addition, more than one of the aforementioned analogues may be incorporated in a single miRNA or even at a single position within an miRNA.

The invention thus provides the insight that an inhibitor of an miRNA present in a platelet, that is over-expressed in a subject suffering from or at risk of suffering from cardiovascular disease, is useful for instance in treating, diminishing, delaying and/or preventing cardiovascular disease.

The invention therefore further provides an inhibitor of an miRNA, said miRNA being selected from the group consisting of miR-545:9.1, miR-624*, and miR-454*, hsa-miR-340*, hsa-miR-615-5p, hsa-miR-451, and homologues and orthologues of any of these miRNAs for use as a medicament. Another embodiment provides an inhibitor of an miRNA, said miRNA being selected from the group consisting of miR-545:9.1, miR-624*, and miR-454*, hsa-miR-340*, hsa-miR-615-5p, hsa-miR-451, and homologues and orthologues of any of these miRNAs for treating, diminishing, delaying and/or preventing cardiovascular disease. In a preferred embodiment, said at least one miRNA is selected from the group consisting of miR-545:9.1, miR-624*, and miR-454*, hsa-miR-340*, hsa-miR-615-5p, and homologues and orthologues of any of these miRNAs. In a more preferred embodiment, said at least one miRNA is selected from the group consisting of miR-545:9.1, miR-624*, miR-454*, and homologues and orthologues of any of these miRNAs. In another more preferred embodiment, said miRNA is hsa-miR-340* or miR-624*, or a homologue or orthologue of hsa-miR-340* or miR-624*.

hsa-miR-340*, hsa-miR-615-5p, hsa-miR-545:9.1, hsa-miR-451, hsa-miR-454*, hsa-miR-624* are all over-expressed in platelets of a subject suffering from or at risk of suffering from a cardiovascular disease. In contrast, the expression of hsa-miR-1280 is lower in platelets of a subject suffering from or at risk of suffering from a cardiovascular disease. For treating, diminishing, delaying and/or preventing cardiovascular disease, hsa-miR-1280 expression or activity must thus be increased.

The invention therefore also provides miRNA hsa-miR-1280 and/or a homologue, orthologue and/or analogue thereof, and/or an activator of hsa-miR-1280 for use as a medicament. In another embodiment the invention provides miRNA hsa-miR-1280 and/or a homologue, orthologue and/or analogue thereof, and/or an activator of hsa-miR-1280 for treating, diminishing, delaying and/or preventing cardiovascular disease.

Also provided is a pharmaceutical composition comprising at least one inhibitor according to the invention and a pharmaceutically acceptable excipient. In a more preferred embodiment, the pharmaceutical composition comprises at least one inhibitor of an miRNA, said miRNA being hsa-miR-340* or a homologue or orthologue thereof. In a more preferred embodiment, the pharmaceutical composition comprises at least one further inhibitor of a miRNA, said miRNA being miR-624* or miR-454* or a homologue or orthologue of mir-624* or miR-454*. In a most preferred embodiment, the pharmaceutical composition comprises at least one inhibitor of miR-624*, or an inhibitor of a homologue or orthologue or mir-624*, at least one inhibitor of hsa-miR-340*, or an inhibitor of a homologue or orthologue or hsa-mir-340*, and at least one inhibitor of miR-454*, or an inhibitor of a homologue or orthologue of miR-454*.

In another more preferred embodiment, the pharmaceutical composition comprises at least one inhibitor of an miRNA, said miRNA being miR-624* or a homologue or orthologue thereof. In a more preferred embodiment, the pharmaceutical composition comprises at least one further inhibitor of a miRNA, said miRNA being hsa-miR-340* or a homologue or orthologue thereof.

Such pharmaceutical compositions according to the invention are especially useful for treating, diminishing, delaying and/or preventing cardiovascular disease. The invention therefore provides a pharmaceutical composition according to the invention for use in treating, diminishing, delaying and/or preventing cardiovascular disease.

The term “cardiovascular disease” encompasses a wide variety of diseases that affect the heart, arteries and veins. According to the invention, a cardiovascular disease is preferably accompanied by or a result of narrowing of an artery. This narrowing can be, but is not necessarily, so severe, that the artery is completely obstructed. The type of artery can be a peripheral artery (e.g resulting in peripheral artery disease of arms or legs, a stroke, kidney failure, or vascular dementia), but also a coronary artery (e.g. resulting in transient ischemic attack, acute coronary syndrome, or stabile angina). In a preferred embodiment, therefore, a method according to the invention, an inhibitor according to the invention, a pharmaceutical composition, and/or an miRNA according to the invention is provided, wherein said cardiovascular disease is selected from the group consisting of atherosclerosis, coronary artery disease, transient ischemic attack, stroke, peripheral vascular disease, acute coronary syndrome, stabile angina, vascular dementia, and kidney failure.

Now that the invention has provided the insight that platelet expression of an miRNA selected from the group consisting of miR-545:9.1, miR-624*, and miR-454*, hsa-miR-340*, hsa-miR-615-5p, hsa-miR-451, and hsa-miR-1280 is predictive for whether a subject is at risk of suffering from a cardiovascular disease, the invention also provides a kit for determining platelet miRNA expression.

In one embodiment, the invention thus provides a kit of parts, comprising at least one molecule capable of specifically binding to at least one miRNA selected from the group consisting of miR-545:9.1, miR-624*, and miR-454*, hsa-miR-340*, hsa-miR-615-5p, hsa-miR-451, hsa-miR-1280, and homologues and orthologues thereof, and an instruction leaflet for use of said kit in a method according to the invention. Said kit may optionally contain one or more controls and/or one or more standards. In a preferred embodiment, said at least one miRNA is hsa-miR-340* or miR-624*, or a homologue, orthologue or analogue of hsa-miR-340* or miR-624*.

In a preferred embodiment, said kit comprises at least two molecules capable of specifically binding to at least two miRNAs selected from the group consisting of miR-545:9.1, miR-624*, and miR-454*, hsa-miR-340*, hsa-miR-615-5p, hsa-miR-451, hsa-miR-1280, and homologues and orthologues thereof. In a more preferred embodiment, said kit comprises at least 3 molecules capable of specifically binding 3, more preferably at least 4 molecules capable of specifically binding 4, more preferably at least 5 molecules capable of specifically binding 5, more preferably at least 6 molecules capable of specifically binding 6, most preferably at least 7 molecules capable of specifically binding 7 miRNAs selected from the group consisting of miR-545:9.1, miR-624*, and miR-454*, hsa-miR-340*, hsa-miR-615-5p, hsa-miR-451, hsa-miR-1280, and homologues and orthologues thereof. It is of course understood that the at least two molecules together bind two miRNAs of the invention, and that each molecule preferably binds only one miRNA. The same holds true for the at least 3, at least 4, at least 5, at least 6, and at least 7 molecules.

In a preferred embodiment, a kit of parts according to the invention is provided, comprising at least two molecules capable of specifically binding at least two miRNAs, wherein a first of said at least two molecules is hsa-miR-340* or a orthologue or homologue of hsa-miR-340*, and a second of said at least two molecules is miR-624*, miR-454*, or a orthologue or homologue of miR-624* or miR-454*.

In a most preferred embodiment, a kit of parts according to the invention is provided comprising at least one molecule capable of specifically binding to hsa-miR-340* and/or to a homologue and/or orthologue of hsa-miR-340*, at least one molecule capable of specifically binding to miR-624* and/or to a homologue and/or orthologue of miR-624*, and at least one molecule capable of specifically binding to miR-454* and/or to a homologue and/or orthologue of miR-454*.

In another embodiment, a kit of parts is provided, comprising at least two molecules capable of specifically binding at least two miRNAs selected from the group consisting of miR-545:9.1, miR-624*, miR-454*, and homologues and orthologues of any of these miRNAs. Even more preferred said kit comprises at least three molecules capable of specifically binding at least three miRNAs selected from the group consisting of miR-545:9.1, miR-624*, miR-454*, and homologues and orthologues of any of these miRNAs.

In yet another embodiment, a kit of parts is provided, comprising at least four molecules capable of specifically binding at least four miRNAs selected from the group consisting of miR-545:9.1, miR-624*, and miR-454*, hsa-miR-340*, hsa-miR-615-5p, hsa-miR-451, hsa-miR-1280, and homologues and orthologues thereof. In a more preferred embodiment, said kit comprises at least 5 molecules capable of specifically binding 5, more preferably at least 6 molecules capable of specifically binding 6, most preferably at least 7 molecules capable of specifically binding 7 miRNAs selected from the group consisting of miR-545:9.1, miR-624*, and miR-454*, hsa-miR-340*, hsa-miR-615-5p, hsa-miR-451, hsa-miR-1280, and homologues and orthologues thereof.

The above mentioned kits are thus useful for determining whether an individual is suffering from, or at risk of suffering from, a cardiovascular disease. In one embodiment, therefore, the use of a kit according to the invention for use in determining whether an individual is suffering from, or at risk of suffering from, a cardiovascular disease is provided.

Various molecules that specifically bind to an miRNA of the invention can be used. Preferably, said at least one molecule is a nucleic acid molecule or an artificial nucleic acid molecule. Nucleic acid molecules are composed of chains of monomeric nucleotides. To improve for instance stability, nucleic acid molecules are artificially modified. Such artificially modified nucleic acid molecules are by definition analogues. Analogues include for instance peptide nucleic acids, as well as nucleic acid sequences comprising at least one modified nucleotide and/or non-natural nucleotide such as for instance inosine, LNA, Morpholino, and 2′-O-methyl RNA.

Nucleic acid molecules capable of specifically binding an miRNA of the invention for use in a kit according to the invention are easily identified by the skilled artisan. A nucleic acid molecule capable of specifically binding to at least one miRNA of the invention preferably comprises a sequence of at least 7 nucleotides with at least 85% complementary sequence identity with said at least one miRNA. In a more preferred embodiment, said nucleic acid molecule comprises at least 8, 9, 10, 11, 12, 13, 14, 15, 16, 17, 18, 19, 20, 21, 22, 23, or at least 24 nucleotides, and has at least 85% complementary sequence identity with said at least one miRNA. In a more preferred embodiment said complementary sequence identity is at least 90%, at least 95%, or 100%. As said before, complementary means the specific pairing of the purines and pyrimidines between the miRNA and the miRNA antisense molecule. With complementary sequence identity is thus meant, the percentage sequence identity between the nucleic acid molecule and the antisense of the miRNA.

Such kit can be provided in the form of an array. Arrays are especially useful for high throughput screening. In yet another embodiment, therefore, the invention provides an array comprising molecules immobilized on a platform, wherein at least one or several of said molecules are capable of specifically binding to at least four miRNA selected from the group consisting of miR-545:9.1, miR-624*, and miR-454*, hsa-miR-340*, hsa-miR-615-5p, hsa-miR-451, hsa-miR-1280, and homologues and orthologues thereof. In another embodiment, the invention provides an array comprising molecules immobilized on a platform, wherein at least one or several of said molecules are capable of specifically binding to at least three, preferably at least four, more preferably at least five, more preferably at least six miRNA selected from the group consisting of miR- 545:9.1, miR-624*, and miR-454*, hsa-miR-340*, hsa-miR-615-5p, hsa-miR-1280, and homologues and orthologues of any of these miRNAs.

In a preferred embodiment, an array according to the invention is provided, wherein at least part of said molecules are capable of specifically binding to hsa-miR-340* and/or to a homologue and/or orthologue of hsa-miR-340*, at least part of said molecules are capable of specifically binding to miR-624* and/or to a homologue and/or orthologue of miR-624*, and at least part of said molecules are capable of specifically binding to miR-454* and/or to a homologue and/or orthologue of miR-454*.

In another embodiment, the invention provides an array comprising molecules immobilized on a platform, wherein at least one or several of said molecules are capable of specifically binding to at least two, preferably at least three, miRNAs selected from the group consisting of hsa-miR-545:9.1, hsa-miR-454*, hsa-miR-624*, and homologues and orthologues thereof. In yet another embodiment, the invention provides an array comprising molecules immobilized on a platform, wherein at least one or several of said molecules are capable of specifically binding to at least two , preferably at least three miRNAs selected from the group consisting of hsa-miR-545:9.1, hsa-miR-454*, hsa-miR-624*, and homologues and orthologues thereof. In a preferred embodiment, a first of said at least two miRNAs is It is for instance possible to synthesize one molecule that is capable of binding several, preferably at least four of said miRNAs. This is for instance achieved by synthesizing a nucleic acid molecule comprising a sequence of at least 7 nucleotides with at least 85% complementary sequence identity with at least one miRNA, further comprising a sequence of at least 7 nucleotides with at least 85% complementary sequence identity with at least one other miRNA, and so forth. It is also possible to synthesize several molecules that each comprise a sequence of at least 7 nucleotides with at least 85% complementary sequence identity with at least one miRNA. Preferably different molecules have at least 85% complementary sequence identity with other miRNA of the invention. In a more preferred embodiment, said nucleic acid molecule(s) comprise/comprises at least 8, 9, 10, 11, 12, 13, 14, 15, 16, 17, 18, 19, 20, 21, 22, 23, or at least 24 nucleotides, and have/has at least 85% complementary sequence identity with said at least one miRNA and/or said at least one other miRNA. In a more preferred embodiment said complementary sequence identity is at least 90%, at least 95%, or 100%.

An array according to the invention preferably comprises molecules, each of which selectively binds an miRNA. The array is preferably accompanied by an instruction leaflet for use of said array in a method according to the invention. As said before, various molecules that specifically bind to different miRNAs of the invention can be used. Preferably, a nucleic acid molecule or an analogue is used in an array according to the invention.

In another preferred embodiment, the plurality of molecules present on said array are capable of specifically binding to at least 5, more preferred at least 6, more preferred at least 7 miRNAs selected from the group consisting of miR-545:9.1, miR-624*, and miR-454*, hsa-miR-340*, hsa-miR-615-5p, hsa-miR-451, hsa-miR-1280, and homologues and orthologues thereof. In another preferred embodiment, the plurality of molecules present on said array are capable of specifically binding to at least four, more preferably at least five, more preferably at least six miRNA selected from the group consisting of miR-545:9.1, miR-624*, and miR-454*, hsa-miR-340*, hsa-miR-615-5p, hsa-miR-1280, and homologues and orthologues of any of these miRNAs.

In one embodiment, the invention provides the use of an array according to the invention in a method according to the invention, preferably for determining whether an individual is suffering from, or at risk of suffering from, a cardiovascular disease.

Before the present invention, several other risk factors were used to determine whether a person suffered from or was at risk of suffering from a cardiovascular disease. These factors include but are not limited to blood pressure, blood cholesterol level, blood glucose level, smoking habit, age, serum C-reactive protein level in said subject, family history, BMI, and/or waist. These risk factors are still useful in addition to the methods according to the invention.

The invention thus provides a method according to the invention, further comprising taking into account blood pressure, blood cholesterol level, blood glucose level, smoking habit, age, serum C-reactive protein level in said subject, family history, BMI, and/or waist for determining the risk on cardiovascular disease. A skilled person is aware of the reference values of these parameters and risks associated with aberrant values.

miRNA levels can be determined by any method known in the art. The skilled person is able to choose a method best suitable for a specific situation. In a preferred embodiment, a method according to the invention is provided, wherein the level of miRNA is detected using a PCR, such as quantitative real-time PCR, microarray, an RNase protection assay, an immunological method, in-situ hybridization, and/or sequencing. These methods are well known in the art and are published in laboratory handbooks, such as for instance in Sambrook's “Molecular Cloning: A Laboratory Manual” (Third Edition, 2001, Cold Spring Harbor Laboratory Press).

Now that the invention has provided the insight that the level of a miRNA of the invention in a platelet is indicative for whether a person is suffering from or at risk of suffering from cardiovascular disease, the invention also provides a method for identifying compounds useful for treating, diminishing, delaying and/or preventing cardiovascular disease. The invention makes use of the insight that expression of an miRNA of the invention is altered in a subject suffering from or at risk of suffering from cardiovascular disease to determine whether candidate compounds are capable of normalizing said expression. With “normalizing” in this context is meant that, if the expression is increased in a subject suffering from or at risk of suffering from cardiovascular disease, the candidate compound is capable of decreasing said expression. Vice versa, a compound is capable of normalizing the expression of an miRNA if the compound is capable of increasing expression of miRNA that is decreased in a subject suffering from or at risk of suffering from cardiovascular disease.

The invention thus provides a method for identifying a compound useful for treating, diminishing, delaying and/or preventing cardiovascular disease, comprising contacting a platelet of a first sample of a subject suffering from or at risk of suffering from cardiovascular disease with a candidate compound, determining a level of expression of at least one miRNA of said platelet of said first sample relative to the expression of at least one miRNA of a platelet of a second sample of said subject which has not been contacted with said candidate compound, said at least one miRNA being selected from the group consisting of miR-545:9.1, miR-624*, and miR-454*, hsa-miR-340*, hsa-miR-615-5p, hsa-miR-451, hsa-miR-1280, and homologues and orthologues of any of these miRNAs, wherein the expression of said at least one miRNA in said first sample, relative to the expression of said at least one miRNA in said second sample is indicative of whether said compound is useful for treating, diminishing, delaying and/or preventing cardiovascular disease. Preferably, said at least one miRNA is selected from the group consisting of miR-545:9.1, miR-624*, and miR-454*, hsa-miR-340*, hsa-miR-615-5p, hsa-miR-1280, and homologues and orthologues of any of these miRNAs. More preferably, said at least one miRNA is selected from the group consisting of miR-545:9.1, miR-624*, miR-454*, and homologues and orthologues of any of these miRNAs. Also preferred is that said at least one miRNA is hsa-miR-340* or a homologue or orthologue of miR-340*.

In a preferred embodiment, a method according to the invention for identifying a compound useful for treating, diminishing, delaying and/or preventing cardiovascular disease is provided, said method further comprising selecting and/or isolating a compound identified as being useful for treating, diminishing, delaying and/or preventing cardiovascular disease.

In a preferred embodiment, a method according to the invention for identifying a compound useful for treating, diminishing, delaying and/or or preventing cardiovascular disease is provided, wherein said at least one miRNA is hsa-miR-340* or a homologue or orthologue of hsa-miR-340*, the method further comprising determining a level of expression of a further miRNA selected from the group consisting of miR-624*, miR-454*, miR-545:9.1, hsa-miR-615-5p, hsa-miR-451, and homologues and orthologues of any of these miRNAs, wherein the level of expression of said hsa-miR-340* or a homologue or orthologue of hsa-miR-340* in combination with the expression of said further miRNA of said second sample, relative to the level of expression of said hsa-miR-340* or a homologue or orthologue of hsa-miR-340* in combination with the expression of said further miRNA of said first sample, is indicative for the effect of said anti-platelet therapy, and wherein said subject has received anti-platelet therapy between said first and said second sample.

In a more preferred embodiment, a method according to the invention for identifying a compound useful for treating, diminishing, delaying and/or or preventing cardiovascular disease is provided, wherein said further miRNA is selected from the group consisting of miR-624*, miR-454*, hsa-miR-451, and homologues and orthologues of any of these miRNAs. In a more preferred embodiment, said further miRNA is miR-454*.

In a most preferred embodiment, a method according to the invention for identifying a compound useful for treating, diminishing, delaying and/or or preventing cardiovascular disease is provided, wherein said further miRNA is miR-624* or a homologue or orthologue of miR-624*, said method further comprising determining a level of expression of miR-454 or a homologue or orthologue of miR-454, wherein the level of expression of said hsa-miR-340* or a homologue or orthologue of hsa-miR-340* in combination with the expression of miR-624* or a homologue or orthologue of miR-624* and in combination with the expression of miR-454 or a homologue or orthologue of miR-454 of said second sample, relative to the level of expression of said hsa-miR-340* or a homologue or orthologue of hsa-miR-340* in combination with the expression of miR-624* or a homologue or orthologue of miR-624* and in combination with the expression of miR-454 or a homologue or orthologue of miR-454 of said first sample, is indicative for the effect of said anti-platelet therapy, and wherein said subject has received anti-platelet therapy between said first and said second sample.

In another preferred embodiment, a method according to the invention for identifying a compound useful for treating, diminishing, delaying and/or or preventing cardiovascular disease is provided, wherein said at least one miRNA is miR-624* or a homologue or orthologue of hsa-miR-624*, the method further comprising determining a level of expression of a further miRNA selected from the group consisting of hsa-miR-340*, miR-454*, miR-545:9.1, hsa-miR-615-5p, hsa-miR-451, and homologues and orthologues of any of these miRNAs, wherein the level of expression of said miR-624* or a homologue or orthologue of miR-624* in combination with the expression of said further miRNA of said second sample, relative to the level of expression of said miR-624* or a homologue or orthologue of miR-624* in combination with the expression of said further miRNA of said first sample, is indicative for the effect of said anti-platelet therapy, and wherein said subject has received anti-platelet therapy between said first and said second sample.

In a more preferred embodiment, a method according to the invention for identifying a compound useful for treating, diminishing, delaying and/or or preventing cardiovascular disease is provided, wherein said further miRNA is selected from the group consisting of hsa-miR-340*, miR-454*, hsa-miR-451, and homologues and orthologues of any of these miRNAs.

It is also possible to monitor the effect of anti-platelet therapy with a method of the invention. Anti-platelet therapy makes use of drugs that interact with platelets to block platelets from aggregating into harmful clots. Anti-platelet drugs include aspirin, ticlopidine (Ticlid®), clopidogrel (Plavix®), tirofiban (Aggrastat®), and eptifibatide (Integrilin®). As said before, a high percentage of persons at risk of suffering from cardiovascular disease do not respond very well on aspirin therapy. With a method according to the invention, such non-responders are identified and can be switched to other medication.

In one embodiment, therefore, the invention provides a method for monitoring the effect of anti-platelet therapy in a subject, the method comprising determining a level of expression of at least one miRNA of a first sample of a subject relative to a level of expression of said at least one miRNA of a second sample of said subject, said at least one miRNA being selected from the group consisting of miR-545:9.1, miR-624*, and miR-454*, hsa-miR-340*, hsa-miR-615-5p, hsa-miR-451, hsa-miR-1280, and homologues and orthologues of any of these miRNAs, wherein the level of expression of said at least one miRNA of said second sample, relative to the level of expression of said at least one miRNA of said first sample, is indicative for the effect of said anti-platelet therapy, and wherein said subject has received anti-platelet therapy between said first and said second sample were taken. Preferably, said at least one miRNA is selected from the group consisting of miR-545:9.1, miR-624*, and miR-454*, hsa-miR-340*, hsa-miR-615-5p, hsa-miR-1280, and homologues and orthologues of any of these miRNAs. More preferably, said at least one miRNA is selected from the group consisting of miR-545:9.1, miR-624*, miR-454*, and homologues and orthologues of any of these miRNAs. In a preferred embodiment, a method according to the invention for monitoring the effect of anti-platelet therapy is provided, wherein said subject is suffering from or is at risk of suffering from cardiovascular disease.

A sample for use in a method according to the invention for monitoring the effect on anti-platelet therapy preferably comprises platelets. It is, however, not necessary that the platelets in the sample are still intact and/or the miRNA is still present within the platelet in order to determine the level of expression thereof. For instance a whole blood sample or a platelet enriched plasma sample can be used in a method according to the invention.

In a preferred embodiment, a method for monitoring the effect of anti-platelet therapy in a subject is provided, wherein said at least one miRNA is hsa-miR-340* or a homologue or orthologue of hsa-miR-340*, the method further comprising determining a level of expression of a further miRNA selected from the group consisting of miR-624*, miR-454*, miR-545:9.1, hsa-miR-615-5p, hsa-miR-451, and homologues and orthologues of any of these miRNAs, wherein the level of expression of said hsa-miR-340* or a homologue or orthologue of hsa-miR-340* in combination with the expression of said further miRNA of said second sample, relative to the level of expression of said hsa-miR-340* or a homologue or orthologue of hsa-miR-340* in combination with the expression of said further miRNA of said first sample, is indicative for the effect of said anti-platelet therapy, and wherein said subject has received anti-platelet therapy between said first and said second sample.

In a more preferred embodiment, a method for monitoring the effect of anti-platelet therapy in a subject is provided, wherein said further miRNA is selected from the group consisting of miR-624*, miR-454*, hsa-miR-451, and homologues and orthologues of any of these miRNAs. In a more preferred embodiment, said further miRNA is miR-454* or a homologue or orthologue thereof.

In a most preferred embodiment, a method for monitoring the effect of anti-platelet therapy in a subject is provided, wherein said further miRNA is miR-624* or a homologue or orthologue of miR-624*, said method further comprising determining a level of expression of miR-454 or a homologue or orthologue of miR-454, wherein the level of expression of said hsa-miR-340* or a homologue or orthologue of hsa-miR-340* in combination with the expression of miR-624* or a homologue or orthologue of miR-624* and in combination with the expression of miR-454 or a homologue or orthologue of miR-454 of said second sample, relative to the level of expression of said hsa-miR-340* or a homologue or orthologue of hsa-miR-340* in combination with the expression of miR-624* or a homologue or orthologue of miR-624* and in combination with the expression of miR-454 or a homologue or orthologue of miR-454 of said first sample, is indicative for the effect of said anti-platelet therapy, and wherein said subject has received anti-platelet therapy between said first and said second sample.

In another preferred embodiment, a method for monitoring the effect of anti-platelet therapy in a subject is provided, wherein said at least one miRNA is miR-624* or a homologue or orthologue of miR-624*, the method further comprising determining a level of expression of a further miRNA selected from the group consisting of hsa-miR-340*, miR-454*, miR-545:9.1, hsa-miR-615-5p, hsa-miR-451, and homologues and orthologues of any of these miRNAs, wherein the level of expression of said miR-624* or a homologue or orthologue of miR-624* in combination with the expression of said further miRNA of said second sample, relative to the level of expression of said miR-624* or a homologue or orthologue of miR-624* in combination with the expression of said further miRNA of said first sample, is indicative for the effect of said anti-platelet therapy, and wherein said subject has received anti-platelet therapy between said first and said second sample.

In a more preferred embodiment, a method for monitoring the effect of anti-platelet therapy in a subject is provided,, wherein said further miRNA is selected from the group consisting of hsa-miR-340*, miR-454*, hsa-miR-451, and homologues and orthologues of any of these miRNAs.

The invention is further illustrated by the following non-limiting examples. The examples do not limit the scope of the invention in any way.

### FIGURE LEGENDS

FIG. 1. Heat map of the supervised hierarchical clustering analysis on the expression profiles of the 24 subjects (sample identification on x-axis). Subjects are labelled as either controls (grey) or cases (black) at the top of the figure. Names of the depicted 214 differentially expressed miRNAs (adjusted P<0.05) are listed in a Table II in the same order. The colour key grey-scale bar indicates standardized miRNA expression levels (dark grey indicates relatively lower expression; light grey indicates relatively higher expression)

FIG. 2. Expression profiles of 7 candidate miRNAs in platelets of CAD patients and control subjects. Left two bars of each figure represent average miRNA expression of 12 healthy controls and 12 CAD patients, as determined by miRNA arrays. Validation by real-time PCR was performed on the same dataset (right two bars). Data are presented as mean ± SEM and *:p<0.05 compared to healthy controls.

FIG. 3. Expression profiles of 6 candidate miRNAs in platelets of CAD patients and control subjects. Left two bars of each figure represent average miRNA expression of the miRNA array cohort (12 controls and 12 patients). Validation by real-time PCR was performed on two independent cohorts (right four bars). Data are presented as mean ± SEM and * indicates p<0.05 compared to healthy controls.

### EXAMPLES

### Example I

### Design and Methods

### Study Population

To create a study population with a genetic predisposition for CADS, we selected a group of 12 Caucasian, male patients with CAD at a young age (premature CAD) and a positive family history of premature cardiovascular disease (CVD). These patients have an ‘extreme-end’ phenotype with a sustained risk for CAD. Therefore, this group is ideal to investigate the role of platelet-specific miRNAs in CAD. The patients were selected from our outpatient clinic of the Academic Medical Centre (AMC) of Amsterdam, which is specialised in premature CAD. The control group was composed of 12 healthy Caucasian male volunteers, which were recruited by advertisement. This control group did not have a history or a positive family history of CVD and was not allowed to use any medication. The study complies with the Declaration of Helsinki, the study protocol was approved by the Medical Ethical Commission of the AMC in Amsterdam and written informed consent was obtained from all subjects.

### Definitions

Premature CAD was defined as a cardiac event before the age of 51 years. A positive family history of premature CVD was defined as one or more 1^{st} degree family member(s) or two or more 2^{nd} degree family members with premature CVD (male <51 years and females <56 years). CAD was defined as either an acute myocardial infarction (AMI) or stable angina pectoris (SAP). AMI was diagnosed clinically by symptoms or electrocardiographic changes, and confirmed by elevated plasma levels of markers of cardiac necrosis. SAP was diagnosed clinically by symptoms and confirmed by significant coronary artery stenosis as shown by coronary angiography. Risk factors were defined in the following manner: hypertension, known treatment for hypertension; diabetes mellitus, known treatment for diabetes mellitus; obesity, body mass index (BMI) >30 kg/m²; hypercholesterolemia, known treatment for hypercholesterolemia.

### Peripheral Blood Collection and Platelet Isolation

Non-fasting venous blood samples were drawn without stasis from an antecubital vein, with use of a 19-gauge needle. Blood was collected in 5 trisodium citrate tubes (each 5 mL containing 0.5 mL 0.105 M trisodium citrate, BD Vacutainer), discarding the first one. Immediately after blood withdrawal, the samples were centrifuged (180 g, 15 min, room temperature, no brake) to obtain platelet-rich plasma (PRP). With a polypropylene pipette, PRP was carefully transferred to a plastic tube leaving at least 25% of the PRP to avoid leukocyte contamination. One part of acid-citrate-dextrose (ACD) buffer (0.085 M trisodium citrate, 0.11 M glucose, 0.071 M Citric acid) was added to five parts of PRP and then the PRP was centrifuged (800 g, 20 min, room temperature, no brake). The platelet-poor plasma was discarded and the platelet pellet carefully resuspended in Tyrode buffer (136.9 mM NaCl, 2.61 mM KCl, 11.9 mM NaHCO₃, 5.55 mM Glucose, 2 mM EDTA, pH 6.5). The platelet suspension was centrifuged (800 g, 20 min, room temperature, no brake). The supernatant was discarded and the platelet pellet was resuspended in 50 μl sterile phosphate buffered saline (PBS) and stored at −80° C. before RNA isolation. The isolated platelets were investigated for contamination by fluorescence-activated cell sorting (FACS) using monoclonal antibodies against CD45-APC (BD Biosciences), CD235a-FITC (DAKO) and CD61-PE (BD Biosciences) to identify respectively leukocytes, erythrocytes and platelets. The purity of the isolated platelets was 99.72%.
RNA Extraction and miRNA Expression Profiling
Total RNA from platelets was extracted using the mirVana PARIS kit (Ambion, Inc.) essentially according to the manufacturer's protocol for liquid samples. The protocol was modified such that samples were extracted twice with an equal volume of acid-phenol chloroform and the column was dried for 3 minutes after the last washing step and before elution. Samples were concentrated from 50 μl to 12 μl, of which 5 μl was used for the array.

MiRNA expression profiles were obtained using Illumina Human v2 MicroRNA BeadArrays according to the manufacturer's recommendation (Illumina, Inc., San Diego, Calif.) at ServiceXS (Leiden, The Netherlands). Raw data were pre-processed, summarized, log-transformed, and quantile normalized using the beadarray package (version 1.12.1) in the statistical software package R (version 2.9.0).

Differential expression was assessed using a moderated t-test using the limma package (version 2.18.3). MiRNAs were considered significantly differentially expressed if the P-values, adjusted for multiple testing by using Benjamini and Hochberg's method, were less than 0.05. Differentially expressed miRNAs (adjusted p<0.05) were visualized by hierarchical clustering of the samples (Euclidean distance, complete linkage). For detailed information see supplementary data online.

Expression analysis by quantitative real time polymerase chain reaction To validate the microarray analysis, differentially expressed miRNAs between patients and controls were selected for real-time PCR. A fixed volume of 8 μl of the eluate from the RNA isolation was used as input in the reverse transcription reaction. Input RNA was reverse transcribed using the miScript reverse transcription kit (Qiagen). The real-time PCR was performed using High Resolution Melting Master (Roche) on a LightCycler480 system II (Roche). Data were analyzed using LinRegPCR quantitative PCR data analysis software, version 11.3.3⁶. After calculation of the NO-values the data were normalized to an endogenous control RNU6B (Applied Biosystems). For detailed information see supplementary data online.

### Results

### Clinical Characteristics

The clinical characteristics of the investigated subjects are shown in Table 1. None of the subjects we're known with diabetes and obesity.

### MiRNA Signatures of Peripheral Platelets

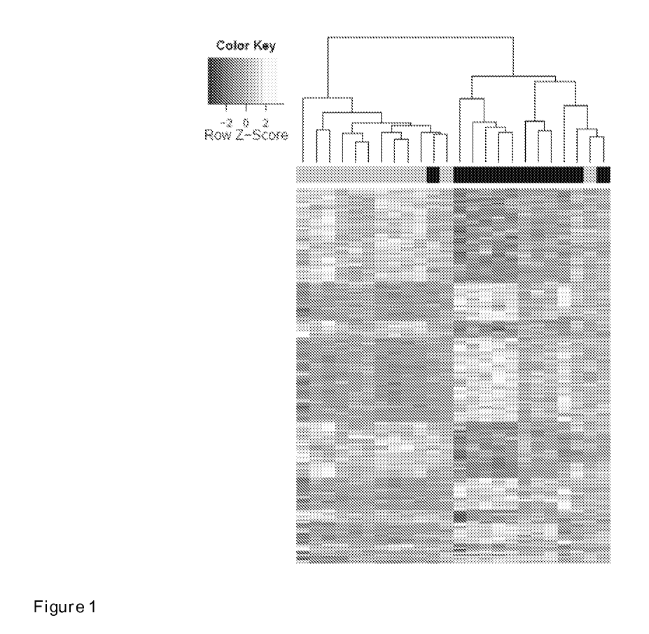

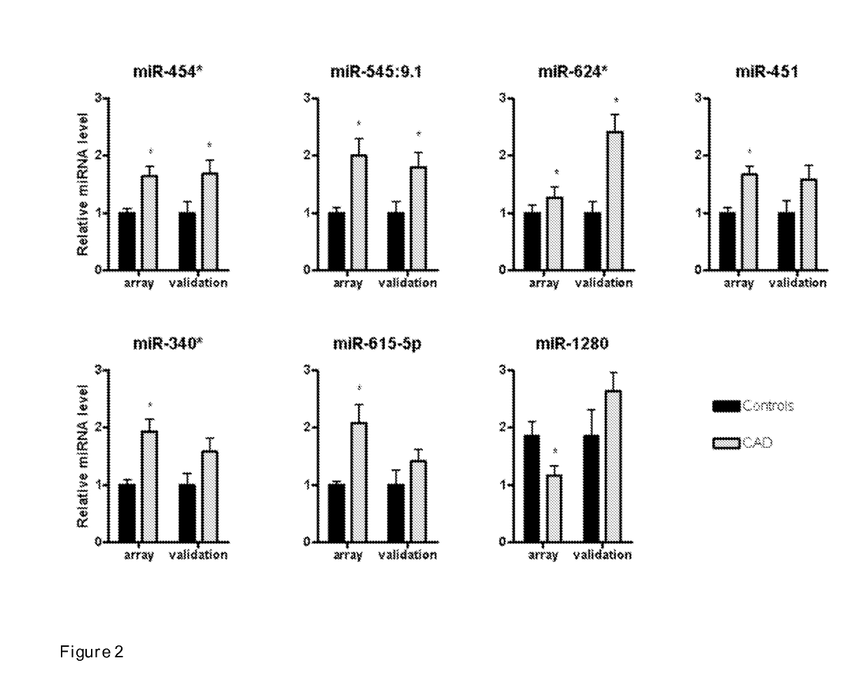

Microarray profiling identified 214 of the 893 detected mature human miRNAs in platelets to be differentially expressed between patients with CAD and healthy controls (adjusted p<0.05). These 214 miRNAs were used in a supervised hierarchical clustering analysis which clusters subjects based on the similarity of their miRNA expression profiles (see FIG. 1). This clustering analysis revealed that a subset of platelet-derived miRNAs could differentiate between CAD patients and healthy controls. From these 214 differentially expressed miRNAs, 9 miRNAs were at least 1.5 fold up-regulated in patients as compared to controls. Furthermore, 4 miRNAs were at least 1.5 fold down-regulated in patients as compared to controls. To validate the robustness of our data, we also analysed our data without the outlying samples mentioned above. Irrespective of which combination of outliers we omitted, 6 miRNAs remained significantly up-regulated and 1 miRNA remained significantly down-regulated. The up-regulated miRNAs were miR-340*, miR-615-5p, miR-545:9.1, miR-451, miR-454* and miR-624*, and the down-regulated miRNA was miR-1280 (see FIG. 2).
Validation of miRNA Array Data by Real-Time PCR
The array data of the 6 up-regulated and 1 down-regulated miRNAs were validated with real-time PCR in the same population. Two control subjects were excluded from the analysis, since the expression levels of the RNU6B (our endogenous control) were consistently out of range as compared to the other samples. All 6 up-regulated miRNAs were shown to be up-regulated by real-time PCR as well, although only three were statistically significant by PCR (miR-545:9.1, miR-624* and miR-454*; p <0.05). The single down-regulated miRNA could not be confirmed (see FIG. 2). PCR conditions and sample size are now optimized in order to confirm the other 3 up-regulated miRNAs and miR-1280.

### EXAMPLE 2

### Design and Methods

### Study Population

### The Validation Cohorts

To confirm the findings of the microarray data, expression levels of selected miRNAs were measured in isolated platelets, in two independent validation cohorts, by qRT-PCR.

### Validation Cohort I

Validation cohort I consisted of 40 premature male CAD subjects and 40 age-matched male controls. These controls were also recruited by advertisement. Participants were selected in the same way as the population used for the miRNA array analysis, using identical inclusion and exclusion and matching criteria. Data collection was done many years after the diagnosis of CAD. Inclusion took place from December 2009 to June 2010. Twenty-seven control subjects were asked to participate in this study twice, to be able to assess miRNA expression before and after medication use. For that matter, they were asked to take acetyl salicylic acid 100mg once daily and simvastatin 40 mg once daily, for 8 weeks.

### Validation Cohort II

Validation cohort II consisted of members of 4 families with high prevalence of premature CAD; 27 atherosclerotic patients and 40 healthy family members. These 4 families were screened at the outpatient clinic. Family members without signs or complaints of CAD underwent a coronary CT-scanning to assess subclinical CAD. Cases were defined as having a history of CAD or a coronary calcium score >80^{th} percentile. Controls were defined without any signs or complaints of CAD and a coronary calcium score <80^{th} percentile.

### Definitions

Premature CAD was defined as a cardiac event before the age of 51 years for males and before the age of 56 for females. A positive family history of premature cardiovascular disease (CVD) was defined as one or more 1^{st} degree family member(s) or two or more 2^{nd} degree family members with premature CVD. CAD was defined as either an acute myocardial infarction (AMI) or stable angina pectoris. AMI was diagnosed clinically by symptoms or electrocardiographic changes and confirmed by elevated plasma levels of markers of cardiac necrosis and confirmed by coronary artery occlusion as shown by coronary angiography single vessel disease. Stable angina pectoris was diagnosed clinically by symptoms and confirmed by significant coronary artery stenosis (>70%) in at least 2 vessels as shown by coronary angiography. Risk factors were defined in the following manner: hypertension: treatment for hypertension or 3 independent measurements of untreated systolic blood pressure >140 mmHg or diastolic blood pressure >90 mmHg on a half hour Dinamap measurement; overweight: BMI>25 kg/m²; hypercholesterolemia: total untreated cholesterol level>8 mmol/l or treatment for hypercholesterolemia before the event; smoking: current smoking; diabetes mellitus: 2 independent measurements of fasting glucose >6.9 mmol/l or non fasting >11.1 mmol/l or known treatment for diabetes mellitus.

All of the above described cohorts comply with the Declaration of Helsinki. The study protocol was approved by the Medical Ethical Commission of the AMC in Amsterdam and written informed consent was obtained from all subjects.

### Peripheral Blood Collection and Platelet Isolation

Non-fasting venous blood samples were drawn without stasis from an antecubital vein, using a 19-gauge needle. Blood was collected in 5 trisodium citrate tubes (each 5 ml containing 0.5 ml 0.105 M trisodium citrate. BD Vacutainer). The first sample was not used for the analysis. Immediately after blood withdrawal, the samples were centrifuged (180 g, 15 min at room temperature with no brake) to obtain platelet-rich plasma (PRP). With a polypropylene pipette, the upper layer of PRP was carefully transferred to a plastic tube to avoid leukocyte contamination. One part of acid-citrate-dextrose (ACD) buffer (0.085 M trisodium citrate. 0.11 M glucose. 0.071 M Citric acid) was added to five parts of PRP and then the PRP was centrifuged (800 g, 20 min at room temperature with no brake). The platelet-poor plasma was discarded and the platelet pellet carefully resuspended in Tyrode buffer (136.9 mM NaCl, 2.61 mM KCl, 11.9 mM NaHCO₃, 5.55 mM Glucose, 2 mM EDTA. pH 6.5). The platelet suspension was centrifuged (800 g, 20 min at room temperature with no brake). The supernatant was discarded and the platelet pellet was resuspended in 50 μl sterile phosphate buffered saline (PBS) and stored at −80° C. prior to RNA isolation. The isolated platelets were investigated by fluorescence-activated cell sorting (FACS) using monoclonal antibodies against CD45 (BD Biosciences), CD235a (DAKO) and CD61 (BD Biosciences) to identify leukocytes, erythrocytes and platelets. The purity of the isolated platelets was 99.72% by FACS analysis.

### RNA Isolation

Platelet RNA was isolated using the mirVana PARIS kit (Ambion. Inc.), according to the manufacturer's protocol for liquid samples. The protocol was modified such that samples were extracted twice with an equal volume of acid-phenol chloroform and the column was dried for 3 minutes after the last washing step and before elution. Samples were concentrated from 50 μl to 12μl, of which 5 μl was used for Illumina arrays as described below.

### Expression Analysis by Quantitative Real-Time PCR

To validate the microarray analysis, differentially expressed miRNAs between patients and controls were selected for real-time PCR. A fixed volume of 8 μl of the eluate from the RNA isolation was used as input in the reverse transcription reaction. Input RNA was reverse transcribed using the miScript reverse transcription kit (Qiagen) or TaqMan MicroRNA reverse transcription kit (Applied Biosystems). The real-time PCR of miR340*, miR451, and miR624* was performed using High Resolution Melting Master (Roche). The real-time PCR of miR454*, miR545:9.1 and miR615-5p were performed by TaqMan MicroRNA assay (Applied Biosystem) and LightCycler 480 probe master (Roche). Both real-time PCR were performed on a LightCycler480 system II (Roche). MiR340*, miR451, and miR624* were analyzed using LinRegPCR quantitative PCR data analysis software version 12.3. MiR454* and miR545:9.1 were analysed using LinRegPCR quantitative PCR data analysis software version 12.5. To date, no normalization protocol has been established to normalize and validate the miRNA content. For this purpose, after calculation of the NO-values data were normalized for both platelet count and miRNA 223, which was similarly expressed throughout all subjects. Results are presented as mean ± SEM using the Statistical Package for the Social Sciences (SPSS) for Windows, version 11.0 (SPSS. Chicago Ill.). Since distribution was not normal, miRNAs were log-transformed. For all of the analyses, a p-value <0.05 was considered to represent a statistically significant difference.

### Results

Validation of the Candidate miRNAs

### Validation Cohort I

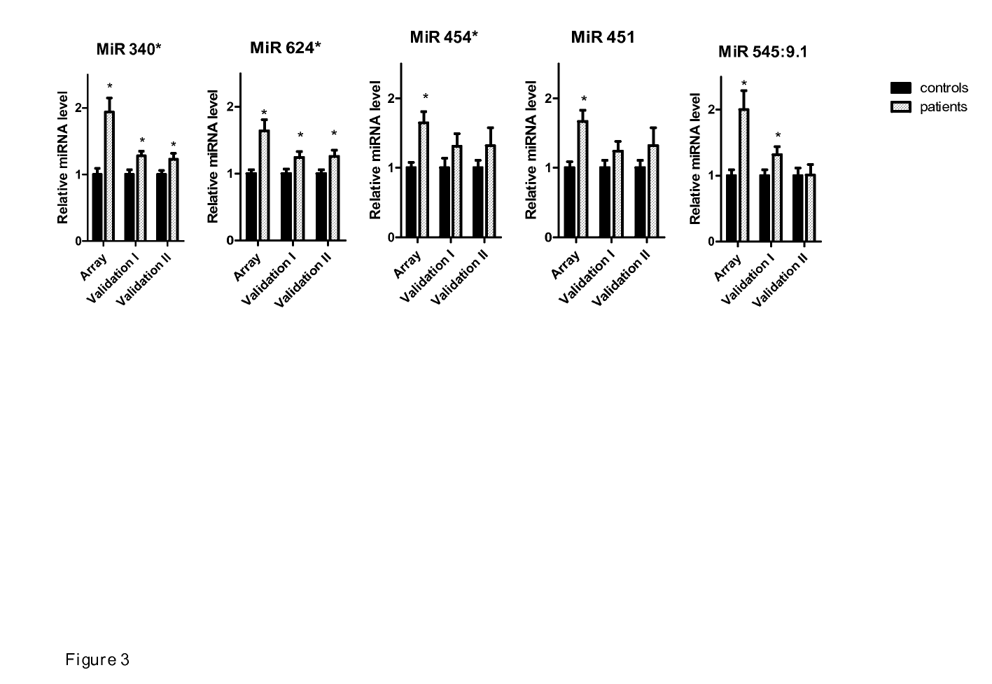

The 7 differentially expressed miRNAs identified by the microarray analysis were first validated, by real-time PCR, in validation cohort I. This cohort consisted of 40 selected patients with premature CAD and 40 healthy matched controls. Of the 7 miRNAs observed in the array analysis, 2 miRNAs, miR340* and miR624*, were significantly up regulated in patients as compared to controls (FIG. 3).

### Validation Cohort II

After the first validation, we validated our results in a second, more general cohort of patients of our premature CAD outpatient clinic. This cohort consisted of 4 families, comprising 27 patients and 40 family members. Of the 7 miRNAs observed in the array analysis, 2 miRNAs, miR340* and miR624*, were significantly up regulated in patients as compared to controls (FIG. 3).
Medication use and Platelet miRNA Expression
To investigate whether medication use might have an influence on the miRNA expression levels of two candidate miRNAs, miR340* and miR624*, the expression of these miRNAs were determined in our control individuals with and without medication, as described in the method section. Interestingly, medication did not change the results.

TABLE 1
Clinical Characteristics

| Clinical | | |
|---|---|---|
| characteristics | Patients (n = 12) | Controls (n = 12) |
| Age at data collection | 46 (6.5) | 45 (7.0) |
| (years, (SD)) | | |
| Age at onset of CAD | 42 (7.6) | not applicable |
| (years, (SD)) | | |
| Currently Smoking | 1 (8) | 1 (8) |
| (number; (%)) | | |
| Hypercholesterolemia | 2 (17) | 0 |
| Hypertension | 2 (17) | 0 |

TABLE 2
Names of the in FIG. 1 depicted 214 differentially
expressed miRNAs (adjusted P < 0.05)

| | |
|---|---|
| 1 | hsa-miR-659 |
| 2 | HS_60 |
| 3 | HS_262.1 |
| 4 | hsa-miR-299-3p |
| 5 | hsa-miR-525-5p |
| 6 | hsa-miR-875-5p |
| 7 | hsa-miR-578 |
| 8 | hsa-miR-1248 |
| 9 | hsa-miR-943 |
| 10 | solexa-539-2056 |
| 11 | hsa-miR-181d |
| 12 | hsa-miR-665 |
| 13 | hsa-miR-206 |
| 14 | hsa-miR-1206 |
| 15 | hsa-miR-1205 |
| 16 | hsa-miR-646 |
| 17 | hsa-miR-367* |
| 18 | HS_268 |
| 19 | HS_177 |
| 20 | hsa-miR-455-3p |
| 21 | hsa-miR-1243 |
| 22 | HS_265.1 |
| 23 | hsa-miR-491-3p |
| 24 | HS_23 |
| 25 | HS_106 |
| 26 | hsa-miR-214* |
| 27 | HS_25 |
| 28 | HS_168 |
| 29 | hsa-miR-548i |
| 30 | hsa-miR-20b* |
| 31 | HS_182.1 |
| 32 | hsa-miR-572 |
| 33 | hsa-miR-106a: 9.1 |
| 34 | hsa-miR-663 |
| 35 | hsa-miR-596 |
| 36 | solexa-15-44487 |
| 37 | hsa-miR-581 |
| 38 | HS_141 |
| 39 | hsa-miR-138-2* |
| 40 | hsa-miR-583 |
| 41 | hsa-miR-641 |
| 42 | hsa-miR-516a-3p, hsa-miR-516b* |
| 43 | hsa-miR-647 |
| 44 | HS_284.1 |
| 45 | hsa-miR-518d-3p |
| 46 | hsa-miR-560: 9.1 |
| 47 | solexa-9578-86 |
| 48 | hsa-miR-614 |
| 49 | hsa-miR-548m |
| 50 | hsa-miR-1231 |
| 51 | HS_40 |
| 52 | hsa-miR-507 |
| 53 | HS_71.1 |
| 54 | hsa-miR-146a* |
| 55 | hsa-miR-520b, hsa-miR-520c-3p, hsa-miR-520f |
| 56 | HS_304_b |
| 57 | HS_267 |
| 58 | hsa-miR-570 |
| 59 | hsa-miR-144 |
| 60 | HS_49 |
| 61 | hsa-miR-648 |
| 62 | hsa-miR-526b |
| 63 | hsa-miR-634 |
| 64 | HS_170 |
| 65 | hsa-miR-1200 |
| 66 | hsa-miR-147 |
| 67 | hsa-miR-1269 |
| 68 | hsa-miR-346 |
| 69 | hsa-miR-1255b |
| 70 | hsa-miR-548c-3p |
| 71 | HS_90 |
| 72 | hsa-miR-1258 |
| 73 | HS_95 |
| 74 | hsa-miR-569 |
| 75 | HS_147 |
| 76 | hsa-miR-100* |
| 77 | hsa-miR-1261 |
| 78 | hsa-miR-195* |
| 79 | HS_79.1 |
| 80 | hsa-miR-298 |
| 81 | hsa-miR-488* |
| 82 | hsa-miR-661 |
| 83 | HS_273 |
| 84 | hsa-miR-636 |
| 85 | hsa-miR-744* |
| 86 | HS_15.1 |
| 87 | hsa-miR-302c |
| 88 | hsa-miR-934 |
| 89 | HS_89 |
| 90 | HS_9 |
| 91 | hsa-miR-1207-3p |
| 92 | hsa-miR-155* |
| 93 | hsa-miR-509-5p |
| 94 | hsa-miR-34c-3p |
| 95 | hsa-miR-455-5p |
| 96 | hsa-miR-551b* |
| 97 | hsa-miR-1283 |
| 98 | solexa-7764-108 |
| 99 | hsa-miR-429 |
| 100 | hsa-miR-920 |
| 101 | hsa-miR-657 |
| 102 | hsa-miR-34a* |
| 103 | hsa-miR-325 |
| 104 | hsa-miR-211 |
| 105 | hsa-miR-31* |
| 106 | hsa-miR-579 |
| 107 | hsa-miR-541 |
| 108 | hsa-miR-1236 |
| 109 | hsa-miR-1278 |
| 110 | hsa-miR-1247 |
| 111 | solexa-3464-254 |
| 112 | hsa-miR-1298 |
| 113 | HS_14.1 |
| 114 | hsa-let-7c* |
| 115 | hsa-miR-1245 |
| 116 | hsa-miR-548g |
| 117 | hsa-miR-1300 |
| 118 | hsa-miR-302c* |
| 119 | hsa-let-7f-2* |
| 120 | hsa-miR-876-5p |
| 121 | hsa-miR-137 |
| 122 | hsa-miR-1299 |
| 123 | hsa-miR-300 |
| 124 | HS_99.1 |
| 125 | hsa-miR-30b* |
| 126 | hsa-miR-922 |
| 127 | HS_176 |
| 128 | hsa-miR-563 |
| 129 | hsa-miR-504 |
| 130 | hsa-miR-9* |
| 131 | HS_122.1 |
| 132 | hsa-miR-1289 |
| 133 | hsa-miR-571 |
| 134 | HS_184 |
| 135 | hsa-miR-1267 |
| 136 | HS_19 |
| 137 | hsa-miR-513a-5p |
| 138 | hsa-miR-591 |
| 139 | HS_110 |
| 140 | hsa-miR-1204 |
| 141 | HS_22.1 |
| 142 | HS_68 |
| 143 | hsa-miR-25* |
| 144 | hsa-miR-516a-5p |
| 145 | hsa-miR-218-1* |
| 146 | solexa-7534-111 |
| 147 | hsa-miR-376a*: 9.1 |
| 148 | hsa-miR-376a* |
| 149 | hsa-miR-299-5p |
| 150 | HS_303_b |
| 151 | HS_157 |
| 152 | hsa-miR-1237 |
| 153 | hsa-miR-550 |
| 154 | hsa-miR-187 |
| 155 | hsa-miR-886-3p |
| 156 | HS_194 |
| 157 | hsa-miR-519b-3p |
| 158 | HS_139 |
| 159 | hsa-miR-1224-3p |
| 160 | hsa-miR-551a |
| 161 | hsa-miR-371-5p |
| 162 | solexa-3022-299 |
| 163 | HS_113 |
| 164 | HS_150 |
| 165 | HS_152 |
| 166 | hsa-miR-10b* |
| 167 | HS_86 |
| 168 | hsa-miR-190 |
| 169 | solexa-3277-272 |
| 170 | hsa-miR-557 |
| 171 | hsa-miR-632 |
| 172 | hsa-miR-1257 |
| 173 | solexa-8926-93 |
| 174 | hsa-miR-1305 |
| 175 | hsa-miR-193a-3p |
| 176 | hsa-miR-362-3p |
| 177 | HS_94 |
| 178 | hsa-miR-374a* |
| 179 | hsa-miR-10a* |
| 180 | hsa-miR-651 |
| 181 | hsa-miR-182* |
| 182 | HS_65 |
| 183 | hsa-miR-615-5p |
| 184 | HS_275 |
| 185 | hsa-miR-181a* |
| 186 | HS_188 |
| 187 | hsa-miR-517a |
| 188 | hsa-miR-615-3p |
| 189 | hsa-miR-17* |
| 190 | hsa-miR-505 |
| 191 | hsa-miR-154 |
| 192 | hsa-miR-454* |
| 193 | HS_303_a |
| 194 | hsa-miR-450b-5p |
| 195 | hsa-miR-624* |
| 196 | hsa-miR-451 |
| 197 | hsa-miR-576-5p |
| 198 | hsa-miR-340* |
| 199 | hsa-miR-335* |
| 200 | hsa-miR-132 |
| 201 | hsa-miR-10a |
| 202 | hsa-miR-585 |
| 203 | hsa-miR-545: 9.1 |
| 204 | hsa-miR-199a-5p |
| 205 | hsa-miR-106b |
| 206 | hsa-miR-1280 |
| 207 | hsa-miR-328 |
| 208 | hsa-miR-151-5p |
| 209 | hsa-miR-23b |
| 210 | hsa-miR-20a |
| 211 | hsa-miR-126* |
| 212 | hsa-miR-374a |
| 213 | hsa-miR-151-3p |
| 214 | hsa-miR-21 |

TABLE 3
AUC and p-values of miRNAs and of combinations of miRNAs

| MIR | (Combined) AUC | p-value |
|---|---|---|
| miR-624* | 0.631 | 0.038 |
| hsa-miR-340* | 0.687 | 0.007 |
| miR-454* | 0.592 | 0.224 |
| hsa-miR-451 | 0.598 | 0.153 |
| 340*, 624* | 0.696 | 0.003 |
| 340*, 454* | 0.712 | 0.001 |
| 340*, 451 | 0.681 | 0.006 |
| 624*, 454* | 0.639 | 0.038 |
| 624*. 451 | 0.618 | 0.077 |
| 451, 454* | 0.618 | 0.076 |
| 624*, 340*, 454* | 0.707 | 0.002 |
| 624*, 340*, 451 | 0.693 | 0.004 |
| 340*, 454*, 451 | 0.710 | 0.002 |
| 624*, 454*, 451 | 0.629 | 0.054 |
| 624*, 340*, 451, 454* | 0.707 | 0.002 |

### References

1. Landry P, Plante I, Ouellet D L, Perron M P, Rousseau G, Provost P. Existence of a microRNA pathway in anucleate platelets. Nat Struct Mol Biol 2009;16:961-6.

2. Ji X, Takahashi R, Hiura Y, Hirokawa G, Fukushima Y, Iwai N. Plasma miR-208 as a biomarker of myocardial injury. Clin Chem 2009;55:1944-9.

3. Ai J, Zhang R, Li Y, Pu J, Lu Y, Jiao J, Li K, Yu B, Li Z, Wang R, Wang L,

Li Q, Wang N, Shan H, Li Z, Yang B. Circulating microRNA-1 as a potential novel biomarker for acute myocardial infarction. Biochem Biophys Res Commun 2010;391:73-7.

4. Wang G K, Zhu J Q, Zhang J T, Li Q, Li Y, He J, Qin Y W, Jing Q. Circulating microRNA: a novel potential biomarker for early diagnosis of acute myocardial infarction in humans. Eur Heart J. 2010 Feb 16. [Epub ahead of print]

5. Scheuner M T. Genetic predisposition to coronary artery disease. Curr Opin Cardiol 2001;16:251-60.

6. Ruijter J M, Ramakers C, Hoogaars W M, Karlen Y, Bakker O, van den Hoff M J, Moorman A F. Amplification efficiency: linking baseline and bias in the analysis of quantitative PCR data. Nucleic Acids Res 2009;37:e45.

1. A method for determining whether a subject is at risk of suffering from a cardiovascular disease, the method comprising determining a (relative) level of expression of at least one miRNA using a sample of said subject, said miRNA being selected from the group consisting of hsa-miR-340*, miR-624*, miR-454*, miR-545:9.1, hsa-miR-615-5p, hsa-miR-451, hsa-miR-1280, and homologues and orthologues of any of these miRNAs, and comparing the level of expression of said at least one miRNA with a reference value, wherein the result of said comparison is indicative of whether said individual is at risk of suffering from a cardiovascular disease.

2. A method according to claim 1, wherein a (relative) level of expression of at least two miRNAs are determined, wherein a first of said at least two miRNAs is hsa-miR-340* or a homologue or orthologue of hsa-miR-340*, and wherein a second of said at least two miRNAs is selected from the group consisting of miR-624*, miR-454*, miR-545:9.1, hsa-miR-615-5p, hsa-miR-451, hsa-miR-1280, and homologues and orthologues of any of these miRNAs, the method further comprising comparing the level of expression of said at least two miRNA with reference values, wherein the result of said comparison is indicative of whether said individual is at risk of suffering from a cardiovascular disease.

3. (canceled)

4. A method according to claim 2, wherein said second miRNA is miR-624* or a homologue or orthologue of miR-624*, said method further comprising determining a (relative) level of expression of miR-454 or a homologue or orthologue of miR-454, and comparing the level of expression of said first, second and further miRNA with reference values, wherein the result of said comparison is indicative of whether said individual is at risk of suffering from a cardiovascular disease.

5. A method according to claim 1, wherein a whole blood sample is used.

6. A method according to claim 1, comprising determining whether the level of expression of said at least one miRNA in said sample is at least 1.50 fold increased relative to the level of expression of said at least one miRNA in a sample of a healthy individual not at risk of cardiovascular disease.

7. A method according to claim 2, comprising determining whether the level of expression of said first and said second miRNA in said sample is at least 1.50 fold increased relative to the level of expression of said first and said second miRNA respectively in a sample of a healthy individual not at risk of cardiovascular disease.

8. A method according to claim 4, comprising determining whether the level of expression of said first and said second miRNA in said sample is at least 1.50 fold increased relative to the level of expression of said first and said second miRNA respectively in a sample of a healthy individual not at risk of cardiovascular disease, and determining whether the level of expression of miR-454 and/or a homologue and/or orthologue of miR-454 in said sample is at least 1.50 fold increased relative to the level of expression of miR-454 and/or a homologue and/or orthologue of miR-454 in a sample of a healthy individual not at risk of cardiovascular disease

9. A method according to claim 4, further comprising determining whether the level of expression of hsa-miR-1280 or a homologue or orthologue thereof in said sample is at least 1.50 fold decreased relative to the level of expression of hsa-miR-1280 or a homologue or orthologue thereof in a sample of a healthy individual not at risk of cardiovascular disease.

10. A method for treating, diminishing, delaying and/or preventing cardiovascular disease, comprising increasing the amount, expression and/or activity of a target of at least one miRNA selected from the group consisting of hsa-miR-340*, miR-624*, miR-454*, miR-545:9.1, hsa-miR-615-5p, hsa-miR-451, and homologues and orthologues of any of these miRNAs in a platelet and/or in a megakaryocyte of a subject suffering from or at risk of suffering from said disease.

11-13. (canceled)

14. A method according to claim 10, wherein said increasing the amount, expression and/or activity of said target of miRNA is mediated through inhibition of the expression or activity of said miRNA in said platelet and/or in said megakaryocyte.

15-16. (canceled)

17. A pharmaceutical composition comprising at least one inhibitor of an miRNA, said miRNA being selected from the group consisting of hsa-miR-340*, miR-624*, miR-454*, miR-545:9.1, hsa-miR-615-5p, hsa-miR-451, and homologues and orthologues of any of these miRNAs and a pharmaceutically acceptable excipient.

18-21. (canceled)

22. A method according to claim 1, wherein said cardiovascular disease is selected from the group consisting of atherosclerosis, coronary artery disease, transient ischemic attack, stroke, peripheral vascular disease, acute coronary syndrome, stabile angina, vascular dementia, and kidney failure.

23-24. (canceled)

25. An array of nucleic acid molecules comprising molecules immobilized on a platform, wherein at least part of said molecules are capable of specifically binding to at least four miRNA selected from the group consisting of hsa-miR-340*, miR-624*, miR-454*, miR-545:9.1, hsa-miR-615-5p, hsa-miR-451, hsa-miR-1280, and homologues and orthologues thereof.

26-27. (canceled)

28. A method for monitoring the effect of anti-platelet therapy in a subject, the method comprising determining a level of expression of at least one miRNA of a first sample of a subject relative to a level of expression of said at least one miRNA of a second sample of said subject, said at least one miRNA being selected from the group consisting of hsa-miR-340*, miR-624*, miR-454*, miR-545:9.1, hsa-miR-615-5p, hsa-miR-451, hsa-miR-1280, and homologues and orthologues of any of these miRNAs, wherein the level of expression of said at least one miRNA of said second sample, relative to the level of expression of said at least one miRNA of said first sample, is indicative for the effect of said anti-platelet therapy, and wherein said subject has received anti-platelet therapy between said first and said second sample.

29-32. (canceled)
